(19) United States
(12) Patent Application Publication
(10) Pub. No.: US 2010/0193674 A1
(43) Pub. Date: Aug. 5, 2010

(54) Lamp system producing uniform high intensity ultraviolet light for exposure of photolithographic and other light polymerizable materials

(75) Inventors: Lionel Fulwood, St. George, UT (US); Greg Baxter, Palm Desert, CA (US)

Correspondence Address: REED SMITH LLP, 101 Second Street, Suite 1800, SAN FRANCISCO, CA, 94105, US

(73) Assignee: WKK Distribution, Ltd.

(21) Appl. No.: 12/322,506

(22) Filed: Feb. 3, 2009

(51) Int. Cl. G01D 18/00; A61N 5/00; H05B 37/00

(52) U.S. Cl. 250/252.1; 250/492.1; 315/312

(57) ABSTRACT

A lamp exposure system producing uniform, high intensity ultraviolet light includes a plurality of lamps generating light in an ultraviolet spectrum range, a separate reflector associated with and covering each of the plurality of lamps, forming distinct lamp and reflector pairs, such that each lamp reflector pair has a generally separate reflective light path to a surface of a substrate, at least one capacitor connected to each lamp to store energy to energize the lamp, and a triggering and control circuit to trigger the at least one capacitor connected to each lamp independently, such that each lamp can be controlled separately with respect to illumination time and intensity. A method for exposing substrates to ultraviolet light, and a method for calibrating the lamp exposure system are also disclosed.

### BACKGROUND OF THE INVENTION

1. Field of the Invention

The present invention relates generally to a system and method for exposing photolithographic materials on various substrates to light, and more particularly, to an improved lamp system for producing high intensity ultraviolet (UV) light for exposure of photolithographic and other light polymerizable materials.

2. Description of the Related Art

In order to “activate” polymers used in photolithographic printed circuit board manufacturing, such as photo-resist polymers or photopolymeric solder masks, ultraviolet light is used. Ultraviolet light is also used in industrial processes to cure or harden various polymerizable materials, such as adhesive layers, cover coats, bonding materials, conformal coatings, and the like. Systems used to generate the ultraviolet light are often referred to as exposure systems or ultraviolet lamp exposure systems.

Current ultraviolet lamp exposure systems often utilize 350-430 nm wavelength lamps that are continually energized during the polymerization cycle. The lamps are usually metal halide or mercury short-arc lamps. A substrate is exposed to the UV light via a mechanical shutter. However, this results in a significant waste of energy at times when no substrate is being exposed. In addition, since the lamps have a limited duty cycle, leaving the lamps on continuously reduces the effective life span of the lamps. These lamps are also limited by the peak energy available, mandating excessively long exposure periods when used with materials requiring a high energy input, such as solder masks, cover coats, conformal coats, etc.

Most materials that are photopolymerized (i.e. light used to form the polymer) require an energy input between 30 and 100 mJ to polymerize them. However, for epoxy type materials, or epoxy acrolates, or other types of materials such as are often used for solder masks on Printed Circuit Boards (PCBs), 400 to 800 mJ or so of energy is normally required. To produce this amount of energy, existing systems often require 30-60 seconds of exposure time per substrate panel. In addition, the lamps are normally placed 1-2 feet from the substrate, resulting in significant energy loss. This lamp location also results in uneven energy distribution across the lamp exposure area, resulting in defective parts.

One prior lamp system for increasing the available energy for polymerization is described in U.S. Patent Application Publication No. 2007/0287091, entitled SYSTEM AND METHOD FOR EXPOSING ELECTRONIC SUBSTRATES TO UV LIGHT, the disclosure of which is herein incorporated by reference. As described, the flash lamp system includes one or more lamps generally configured to produce light on a substrate via a single, common reflector assembly. In other words, the lamps operate over a common reflective light path. While this reference does discuss triggering the lamps at different times, there is no way to control the specific location or intensity of the light energy at any particular area on the exposure surface, since the lamps generally share the common light path. Also, since the lamps are located away from the surface, more energy is required to trigger the lamps and hence large capacitors are required.

Thus, there is a need in the art for an improved lamp system for providing ultraviolet light for use in polymerizing various materials.

### SUMMARY OF THE INVENTION

In general, the present invention is a lamp exposure system producing uniform, high intensity ultraviolet light. According to one embodiment, a lamp system comprises a plurality of lamps generating light in an ultraviolet spectrum range, a separate reflector associated with and covering each of the plurality of lamps, forming distinct lamp and reflector pairs, such that each lamp reflector pair has a generally separate reflective light path to a surface of a substrate, at least one capacitor connected to each lamp to store energy to energize the lamp, and a triggering and control circuit to trigger the at least one capacitor connected to each lamp independently, such that each lamp can be controlled separately with respect to illumination time and intensity.

According to another embodiment a lamp exposure system for exposing a substrate to ultraviolet light comprises a plurality of ultraviolet lamps, a separate reflector associated with and covering each of the plurality of lamps, forming distinct lamp and reflector pairs, such that each lamp reflector pair has a generally separate reflective light path to a surface of a substrate, wherein the plurality of lamps and reflectors are arranged in a rectangular array, such that each lamp is separated from adjacent lamps by respective reflectors, and the light energy from each lamp is independently directed to a different area of the substrate, and wherein the rectangular array is located less than 6 inches from the surface of the substrate, at least one capacitor connected to each lamp to store energy to energize the lamp, and a triggering and control circuit to flash trigger the at least one capacitor connected to each lamp independently, such that each lamp can be controlled separately with respect to illumination time and intensity.

A method of exposing a substrate to ultraviolet light according to the present invention comprises measuring light energy from each of a plurality of ultraviolet lamps in order to normalize the intensity of light energy across a surface of a substrate, wherein each lamp is directed toward a different surface area of the substrate, determining a triggering profile for each lamp based on a result of the measuring, storing each triggering profile in a memory, placing a substrate within ten inches of the plurality of ultraviolet lamps, wherein each lamp has an associated reflector, independently flash triggering each lamp according to its stored triggering profile in at least a subset of the plurality of ultraviolet lamps for a predetermined period of time.

A method of calibrating a lamp exposure system having a plurality of ultraviolet lamps comprises measuring light energy at a plurality of positions across a surface of a substrate located a fixed distance from the plurality of ultraviolet lamps, determining a triggering profile for each lamp based on the measuring, in order to produce a uniform energy intensity across the surface of the substrate, and storing the triggering profile for each lamp.

The step of measuring may comprise placing a radiometer at a first position and measuring the light energy, and moving the radiometer to a next position and measuring the light energy until each position has been measured, placing a test bed comprising a plurality of radiometers at the substrate location and measuring the light energy at each position and/or exposing a test board having a standardized gray scale polymer pattern at fixed positions to the plurality of ultraviolet lamps; and evaluating the gray scale pattern at each fixed position to determine a level of light energy at each position.

### BRIEF DESCRIPTION OF THE DRAWINGS

The present invention will be readily understood by the following detailed description in conjunction with the accompanying drawings, wherein like reference numerals designate like structural elements, and in which:

FIG. 1 is a diagram of one embodiment of a lamp exposure system according to the present invention;

FIG. 2 is a backside view of the lamp exposure system of FIG. 1, utilizing a computer controller;

FIG. 3 is a diagram of an embodiment of a reflector;

FIG. 4 is diagram of a preferred lamp flash curve, according to one embodiment of the invention;

FIG. 5 is a schematic of the wiring of a lamp exposure system according to one embodiment of the present invention;

FIGS. 6A-6D is a schematic of the wiring of the lamps according to one embodiment of the present invention; and

FIG. 7 is a flowchart of the operation of the lamp exposure system, according to one embodiment of the present invention.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention.

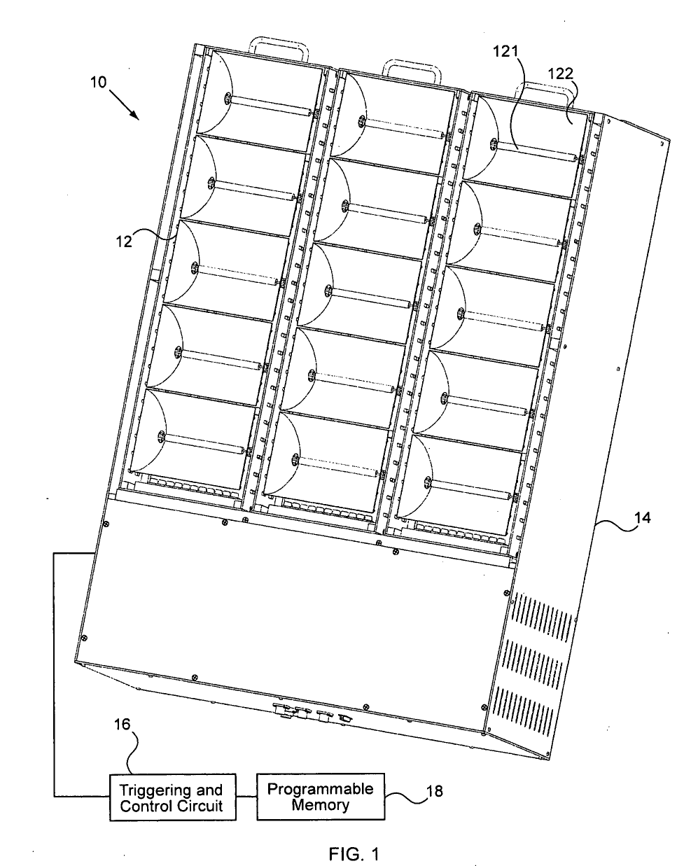

An embodiment of the present invention is illustrated in FIG. 1. As shown, a lamp exposure system 10 according to the present invention includes a flash lamp exposure module 12 of high intensity ultraviolet lamps arranged in a 3×5 rectangular array, a bank of capacitors 14 to energize the lamps 12, and a triggering and control circuit 16 to control the triggering of the lamps.

The flash lamp module 12 comprises 15 lamps (i.e. lamp 121) arranged in a 3 by 5 array (grid). The present description is directed to this specific embodiment having an array of 15 lamps, however, the number of lamps can be more or less, without departing from the teachings of the present invention. In addition, the lamps can be bigger or smaller than those described herein, and have greater or less energy output. Furthermore, the present invention is described with respect to a preferred embodiment for use with PCBs, but the teachings are applicable to other substrates having polymerizable or photo-curable material.

With respect to the embodiment in FIG. 1, each lamp covers an area of about 6″×8″ (150 mm×200 mm) and, much like pixel arrangements on a display, are positioned together to form the basis to provide intense and highly uniform ultraviolet light to a substrate. Each lamp has its own reflector (i.e. reflector 122), which generally surrounds the lamp on four sides, and separates each lamp from the adjacent lamps. Each reflector thereby forms a generally unique reflective light path to the surface of a substrate (not shown) for each lamp.

In a preferred configuration, a substrate having polymer material to be processed is located within approximately 10 inches (250 mm) of the lamp, or approximately six inches (150 mm) from the reflector edges. In such a configuration, some light from an adjacent lamp will affect the energy intensity under each adjacent lamp, but generally the energy intensity at any point on the surface of the substrate will be most directly determined by the lamp positioned directly over the particular point on the substrate.

Thus, the present configuration allows for greater control of the energy intensity at any given position on the substrate surface, compared with the prior designs in which the lamp(s) shared a common light path and a single reflector configuration. In addition, by placing the lamps closer to the substrate than prior systems, lower power lamps, smaller capacitors and/or less power can be used to achieve the same light intensity on the surface of the substrate.

A bank of capacitors 14 is used to provide the requisite voltage and current to energize each ultraviolet lamp. In this embodiment two separate capacitors are connected to each lamp, such that for 15 lamps, 30 capacitors are used. In this embodiment, the capacitors are approximately 1200 microfarads, and the lamps function similarly to PerkinElmer® DG 8901-1 type flash lamps. The lamps are preferably treated to prevent the generation of ozone during use.

The system includes a triggering and control circuit 16 to control the triggering of the lamps 12. In one embodiment, the lamps 12 can all be energized in unison. However, a unique advantage of the present invention is the ability to independently control each lamp. In other words, the triggering and control circuit 16 can separately activate each lamp by separately controlling in the discharge of each lamp's respective capacitors. The lamps 12 can thus be turned on and off independently of any of the other lamps. This allows the lamps to be energized according to a desired sequence and provides the ability to only energize a “sub-array” of the lamps.

The triggering and control circuit 16 is programmable, and different process controls with respect to timing, sequencing, duty cycle, etc. can be stored in a programmable memory, along with a specific triggering profile (described below) for each lamp. The triggering and control circuit 16 may be formed as an ASIC (Application Specific Integrated Circuit), a custom programmed circuit, or as a standard general purpose computer, as in known in art.

The ability to program the total energy output from individual lamps also allows those skilled in the art to provide for the normal lamp degradation resulting in a reduced total energy output. Lamps tend to degrade differently, and so there might be a marked difference between lamps within the array as they degrade with time. The programmable aspect of the control system allows the operator to “tune” each lamp to provide the desired energy output.

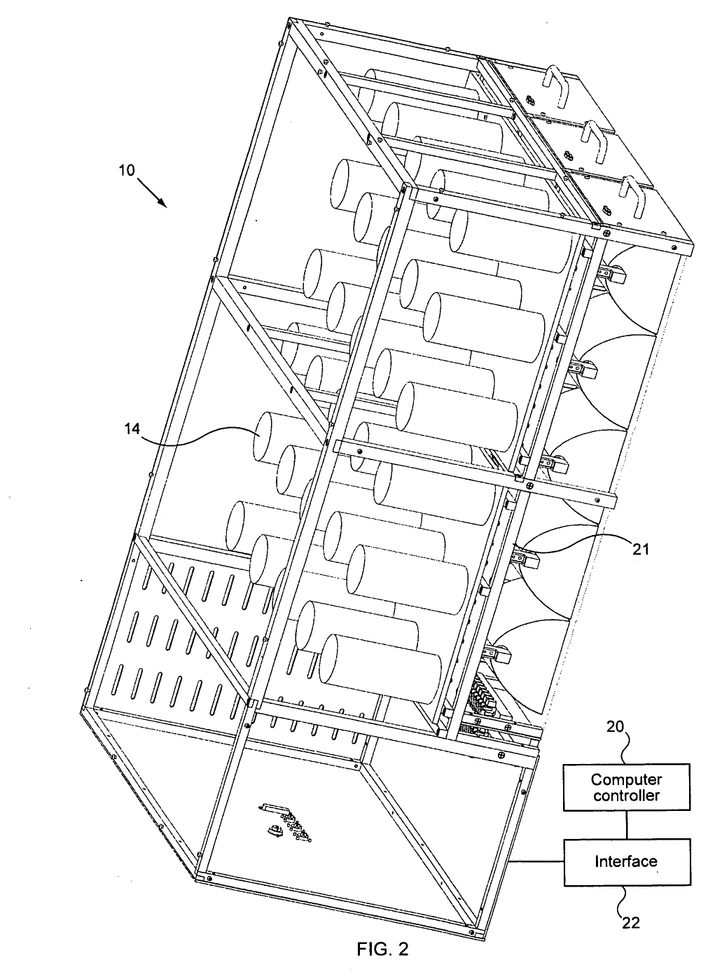

FIG. 2 is a backside view of a light exposure system according to the present invention. As shown in FIG. 2, in a preferred embodiment, the triggering and control circuit and programmable memory is implemented using a standard personal computer 20 programmed to control the lamps, and connected to the light exposure system using a standard industrial control interface 22, as is well known in the art. Depending on the desired configuration and the design of the lamp exposure interface, the control interface 22 may not be necessary. According to one embodiment, an interface card to a PC can be formed using a Complex Programmable Logic Device (CPLD). This CPLD hosts all the timing and sequencing schemes. Under control of a PC, the CPLD sends out the necessary control signals to the lamp system.

While the present invention has been described with respect to a single array of lamps 12, two arrays of lamps can be utilized and positioned on the top and bottom of a substrate to be processed. The two light arrays could each have its own separate bank of capacitors and control circuitry, or both light arrays can be energized and controlled by common components.

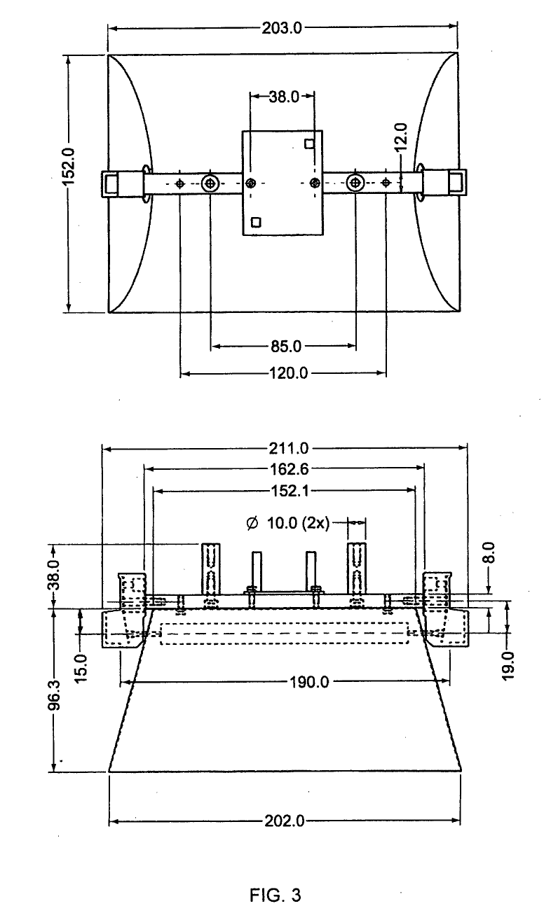

In one embodiment, the lamp module is an approximately 20″×30″ (508 mm×762 mm) array that provides a 24″×30″ (610 mm×762 mm) image area. FIG. 3 illustrates the dimensions of an individual reflector element (i.e. reflector 122).

As noted above, one common use for the light exposure system of the present invention is to polymerize photo-sensitive solder mask materials on Printed Circuit Boards (PCBs). Such photo-polymers require high levels of energy for exposure. In order to automate the production of PCBs, a great deal of ultraviolet power is necessary, and the energy is preferably uniformly distributed across the surface of substrate to insure the correct geometry and degree of polymerization. Accordingly, the light output for each lamp of the array is adjusted to provide optimum uniformity of the exposure energy.

More particularly, the types of high intensity ultraviolet lamps used in previous lamp exposure systems typically have an “always on” life cycle of about 1000 hours. Thus, the prior art systems would be required to regularly replace the lamps every 600-1000 hours or so. However, each new lamp does not output the exact same energy level, even with the same input. Also, as a lamp ages, its effective energy output deceases. Finally, different polymerizable materials require a different amount of energy to effectively polymerize.

Prior art systems lack an effective method to control these variations in energy intensity, especially over time. In addition, with only one common reflective light path in the prior art system, there is no way to adjust the lamp(s) to create a more uniform intensity across the surface of a substrate to be processed as the lamps degrade.

However, since the present invention can control each lamp individually, the deficiencies of the prior art can be overcome. In the present system, the light output for each lamp of the array is adjusted to provide optimum uniformity of the exposure energy. Specifically, the combination of a plurality of smaller lamps arranged in an array, with individual lamp control, provides a greatly improved system.

According to an embodiment of the present invention, the energy output of each lamp in the array is measured and compared to a standard baseline. One approach to do this is to use a standard PCB substrate having one or more industry standard “gray scale” photo-tools applied to the board. The test board can be formed with multiple gray scale patterns arranged at fixed positions, or a single small board can be used and placed at each lamp position (requiring multiple boards). The gray scale provides a measure of the amount of polymerization of the photo-polymer. Similarly, a UV radiometer can be placed at various locations under the lamps in the array, or a test bed of multiple radiometers can be made to measure the light intensity at different locations. Based on this data, it can be determined whether each lamp is operating above, below, or at the desired baseline intensity.

The light intensity data can be entered into the triggering and control circuit and/or computer. For a fully automated system, the output of a radiometer test bed can be fed directly into the control computer. Based on the data obtained, the triggering and control circuit (or software running on a computer) determines which lamps need to operate at a relative higher power output, which ones at a relatively lower output, etc. The triggering and control circuit and/or software then stores a “triggering profile” for each lamp in the system. The triggering profile will control the amount of charge each capacitor stores/discharges on each triggering cycle, thereby controlling the amount of light energy output by each respective lamp. For example, the triggering profile can include a time duration value to normalize a particular lamp's output energy with a baseline flash energy output.

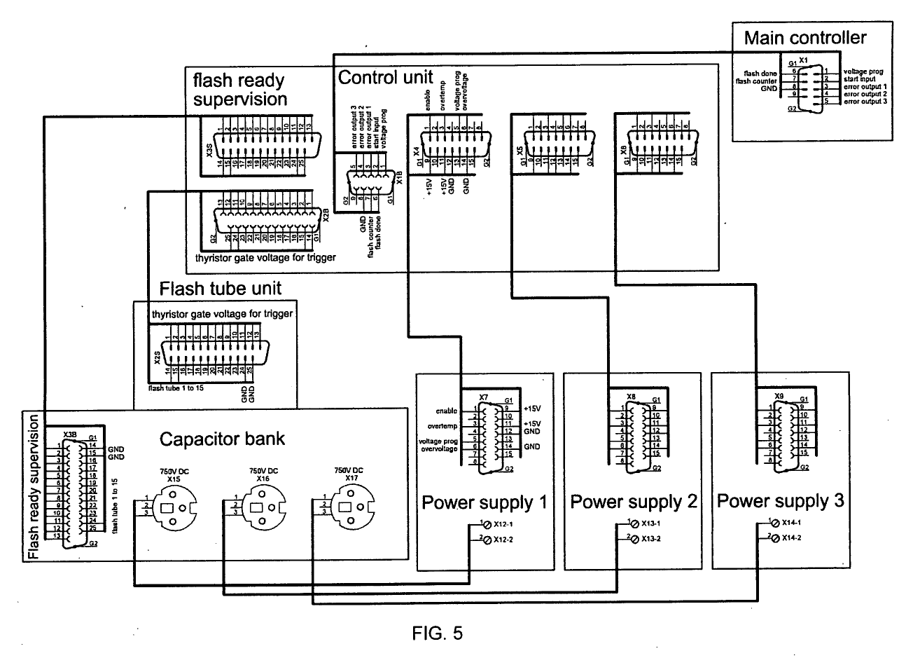

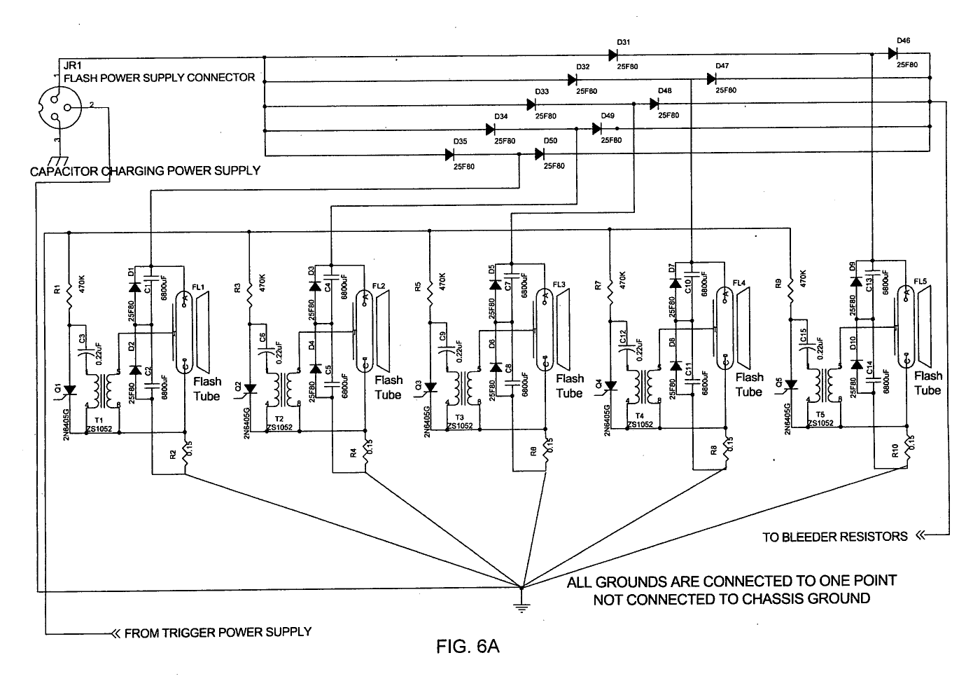

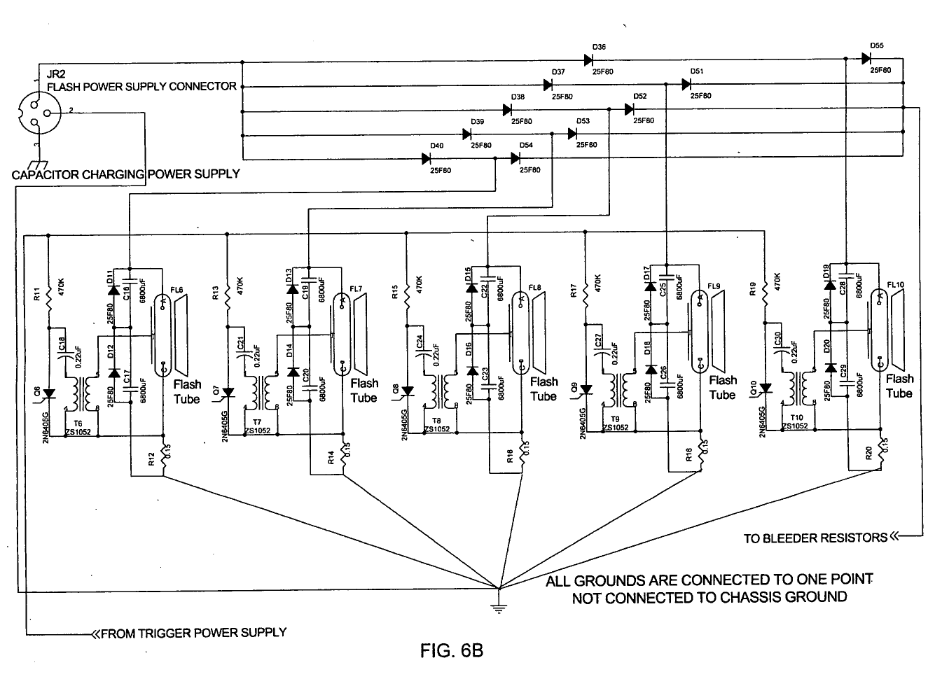

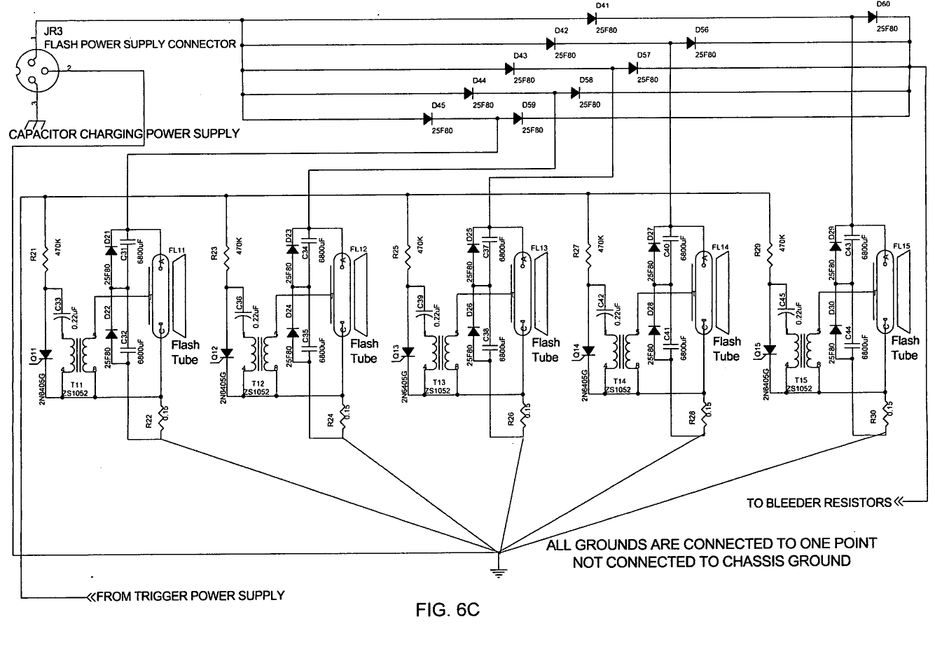

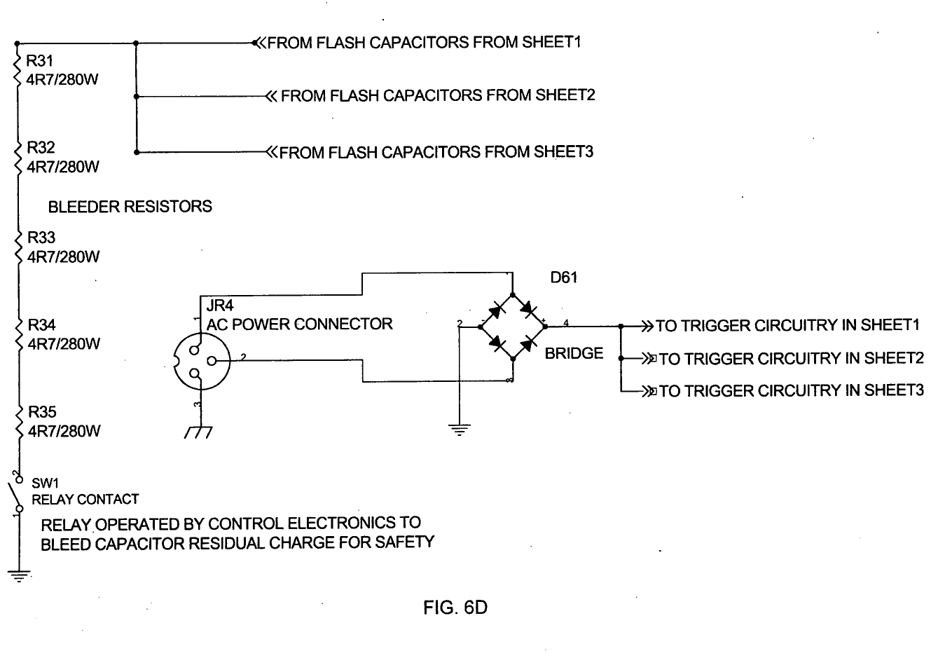

As noted above, the lamp exposure system is preferably controlled using a programmed computer (as shown in FIG. 2). FIG. 5 is a schematic view of the wiring connections between the various modules according to a preferred embodiment. FIGS. 6A-6D is a schematic of the wiring of each of the lamps in greater detail. The “main controller” module connects the controller I/O lines to an external PC for programmable control of the lamp exposure system.

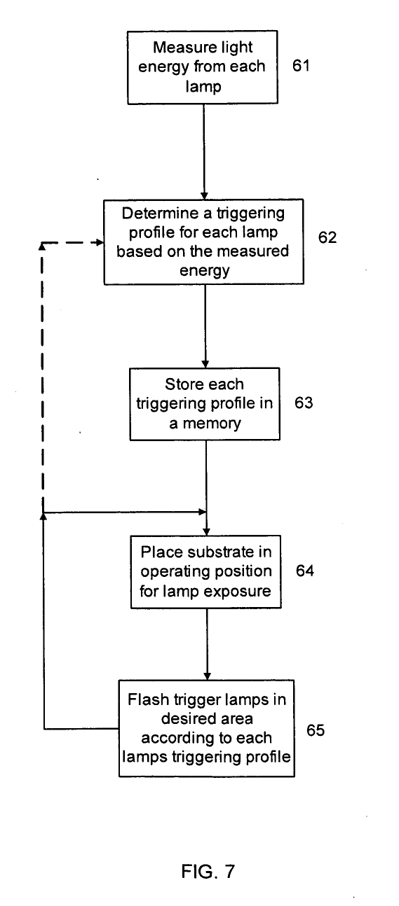

FIG. 7 is a flowchart illustrating the operation according to one embodiment of the present lamp exposure system. At step 61, the light energy output from each lamp is measured, as described above. Based on the measured energy level, a triggering profile is determined for each lamp (step 62), and the triggering profile is stored in memory (step 63). This procedure can be performed at regular intervals, such as daily or weekly, to calibrate the system. Next, a substrate, such as PCB having a photo-resist polymer material on it is placed into position for exposure (step 64). The ultraviolet lamps are flash triggered according to each lamps triggering profile (step 65) and/or the pre-programmed exposure settings. The loading and processing steps repeat for subsequent substrates, and periodically (i.e. daily, each shift change, etc.), the system is re-calibrated to adjust the triggering profiles for the lamps to account for lamp degradation.

In order to increase the lifespan of the lamps, one technique is to power a lamp only to 80% or so of its maximum light output. For applications where 80% of the maximum provides sufficient energy output, the power to the lamps can be increased over time to compensate for the reduced power output caused by aging. This both reduces the average energy usage of the system, and increases the life span of the lamps.

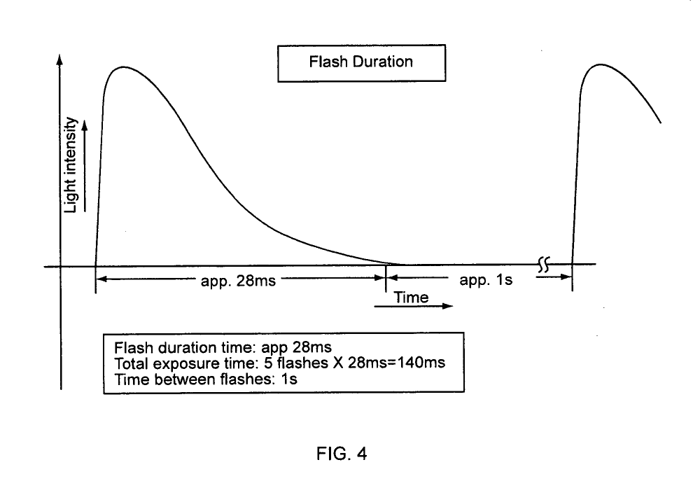

The present invention further reduces the energy consumption of a lamp exposure system by “flash triggering” the lamps, and placing the lamps in close proximity (less than 10 inches) to the substrate. Specifically, the lamps are energized only when a substrate is being exposed. For example, in the case of a PCB with a photo-resist polymer requiring a high level of energy, the lamps can be “flashed” five times in five seconds. As shown in FIG. 4, a preferred embodiment is to flash each lamp for approximately 28 milliseconds, with approximately a second between flashes, for a total time of approximately five seconds. The total exposure time is approximately (5×28) 140 milliseconds.

If it is assumed that when the lamp exposure system is integrated into an automatic exposure machine, the machine can unload an exposed substrate and load a new substrate in approximately fifteen seconds, the total duty time for a processing cycle is twenty seconds. This is a tremendous speed and efficiency improvement over prior systems which could take 30-60 seconds just to expose one substrate. Also, since the lamps are operating with less than a 25% duty cycle (the lamps are actually “on” for only a fraction of 5 out of 20 total seconds), the present lamp exposure system produces much less heat than prior systems. This is important for controlling the thermal coefficient of expansion for materials in the lamp exposure system, especially with respect to polyester templates (artwork) used in photolithography. Specifically, the lower average operating temperature reduces the expansion/variation of the artwork, thus providing better registration and alignment of the artwork and the PCB, thereby increasing production yields.

Another advantage of the present design is that less than all of the lamps in the lamp module can be used for a given application. Using the example of an array module having 15 lamps, a standard 24″×30″ PCB can be exposed at once by using all the lamps. However, if the desired board for processing is only 16″×18″, then only 6 of the lamps can be used (i.e. a sub-array). Since the triggering and control circuit and/or control computer can easily trigger only the desired lamps, any combination of lamps can be used as desired for a specific application. This reduces the power consumption for smaller substrates, and provides greater system flexibility.

Those skilled in the art will appreciate that various adaptations and modifications of the just-described preferred embodiments can be configured without departing from the scope and spirit of the invention. Therefore, it is to be understood that, within the scope of the appended claims, the invention may be practiced other than as specifically described herein.

What is claimed is:

1. A lamp system comprising:
a plurality of lamps generating light in an ultraviolet spectrum range;
a separate reflector associated with and covering each of the plurality of lamps, forming distinct lamp and reflector pairs, such that each lamp reflector pair has a generally separate reflective light path to a surface of a substrate;
at least one capacitor connected to each lamp to store energy to energize the lamp; and
a triggering and control circuit to trigger the at least one capacitor connected to each lamp independently, such that each lamp can be controlled separately with respect to illumination time and intensity.

2. The lamp system of claim 1, wherein the lamps are positioned less than 10 inches from a substrate.

3. The lamp system of claim 1, wherein each lamp is flash triggered independently of the other lamps.

4. The lamp system of claim 3, wherein each lamp is flash triggered for a period of up to 5 seconds.

5. The lamp system of claim 1, wherein each lamp is flashed 5 times within 5 seconds, and each flash is approximately 28 milliseconds in duration.

6. The lamp system of claim 5, wherein the system has a 20 second duty cycle, such that the lamps are energized only during a period of 5 seconds, and are off for a period of 15 seconds.

7. The lamp system of claim 1, wherein the plurality of lamps and reflectors are arranged in a first rectangular array, such that each lamp is separated from adjacent lamps by respective reflectors, and the light energy from each lamp is independently directed to a different area of a substrate.

8. The lamp system of claim 7, wherein only a sub-region of the lamps in the first rectangular array are energized.

9. The lamp system of claim 8, wherein the lamps are triggered at different times, according to a programmed sequence.

10. The lamp system of claim 8, wherein the triggering and control circuit increases the illumination intensity for each lamp, as each lamp ages.

11. The lamp system of claim 7, further comprising a second rectangular array of lamps and reflectors located on an opposite side of a substrate, wherein the triggering and control circuit controls the lamps in the second rectangular array independently of the lamps in the first rectangular array.

12. The lamp system of claim 7, wherein the triggering and control circuit comprises a programmable memory to store a triggering profile for each lamp.

13. The lamp system of claim 7, wherein the triggering and control circuit comprises a computer programmed to trigger and control the at least one capacitor connected to each lamp according to a predefined triggering profile for each lamp.

14. The lamp system of claim 13, wherein the triggering profile for each lamp is periodically updated based on results of a system calibration.

15. A lamp exposure system for exposing a substrate to ultraviolet light, the system comprising:
a plurality of ultraviolet lamps;
a separate reflector associated with and covering each of the plurality of lamps, forming distinct lamp and reflector pairs, such that each lamp reflector pair has a generally separate reflective light path to a surface of a substrate, wherein the plurality of lamps and reflectors are arranged in a rectangular array, such that each lamp is separated from adjacent lamps by respective reflectors, and the light energy from each lamp is independently directed to a different area of the substrate, and wherein the rectangular array is located less than 6 inches from the surface of the substrate;
at least one capacitor connected to each lamp to store energy to energize the lamp; and
a triggering and control circuit to flash trigger the at least one capacitor connected to each lamp independently, such that each lamp can be controlled separately with respect to illumination time and intensity.

16. The lamp exposure system of claim 15, wherein at least some of the lamps in the rectangular array are flash triggered each exposure cycle, and the substrate is exposed to ultraviolet light during a period of approximately 1-5 seconds.

17. A method of exposing a substrate to ultraviolet light, the method comprising:
measuring light energy from each of a plurality of ultraviolet lamps in order to normalize the intensity of light energy across a surface of a substrate, wherein each lamp is directed toward a different surface area of the substrate;
determining a triggering profile for each lamp based on a result of the measuring;
storing each triggering profile in a memory;
placing a substrate within ten inches of the plurality of ultraviolet lamps, wherein each lamp has an associated reflector;
independently flash triggering each lamp according to its stored triggering profile in at least a subset of the plurality of ultraviolet lamps for a predetermined period of time.

18. A method of calibrating a lamp exposure system having a plurality of ultraviolet lamps, the method comprising:
measuring light energy at a plurality of positions across a surface of a substrate located a fixed distance from the plurality of ultraviolet lamps;
determining a triggering profile for each lamp based on the measuring, in order to produce a uniform energy intensity across the surface of the substrate; and
storing the triggering profile for each lamp.

19. The method of claim 18, wherein the step of measuring comprises placing a radiometer at a first position and measuring the light energy, and moving the radiometer to a next position and measuring the light energy until each position has been measured.

20. The method of claim 18, wherein the step of measuring comprises placing a test bed comprising a plurality of radiometers at the substrate location and measuring the light energy at each position.

21. The method of claim 18, wherein the step of measuring comprises:
exposing a test board having a standardized gray scale polymer pattern at fixed positions to the plurality of ultraviolet lamps; and
evaluating the gray scale pattern at each fixed position to determine a level of light energy at each position.
